(19) United States
(12) Patent Application Publication
(10) Pub. No.: US 2020/0069291 A1
(43) Pub. Date: Mar. 5, 2020

(54) METHODS AND APPARATUSES FOR COLLECTION OF ULTRASOUND DATA

(71) Applicant: Butterfly Network, Inc., Guilford, CT (US)

(72) Inventor: Maxim Zaslavsky, San Diego, CA (US)

(73) Assignee: Butterfly Network, Inc., Guilford, CT (US)

(21) Appl. No.: 16/553,693

(22) Filed: Aug. 28, 2019

Related U.S. Application Data
(60) Provisional application No. 62/724,466, filed on Aug. 29, 2018.

(51) Int. Cl. A61B 8/00; G09B 19/24; G09B 19/00

(52) U.S. Cl. CPC A61B 8/46; G09B 19/003; G09B 19/24; A61B 8/4472

(57) ABSTRACT

Aspects of the technology described herein relate to instructing a user to use an ultrasound device to collect ultrasound data. A local processing device may provide an instruction to collect sets of data from multiple positions of the ultrasound device relative to a subject. The local processing device may receive sets of data from the ultrasound device, each of the sets of data including ultrasound data collected at a particular position of the ultrasound device relative to the subject. The local processing device may transmit the sets of data to a remote processing device. The local processing device may receive, from the remote processing device, an indication of a selected set of data from among the sets of data. The local processing device may provide an instruction to move the ultrasound device to the position of the ultrasound device at which the selected set of data was collected.

### CROSS-REFERENCE TO RELATED APPLICATIONS

The present application claims the benefit under 35 U.S.C. § 119(e) of U.S. Patent Application Ser. No. 62/724,466, filed Aug. 29, 2018 under Attorney Docket No. B 1348.70100US00, and entitled “METHODS AND APPARATUSES FOR COLLECTION OF ULTRASOUND DATA,” which is hereby incorporated herein by reference in its entirety.

### FIELD

Generally, the aspects of the technology described herein relate to ultrasound data collection. Some aspects relate to instructing a user to use an ultrasound device to collect ultrasound data.

### BACKGROUND

Ultrasound probes may be used to perform diagnostic imaging and/or treatment, using sound waves with frequencies that are higher than those audible to humans. Ultrasound imaging may be used to see internal soft tissue body structures, for example to find a source of disease or to exclude pathology. When pulses of ultrasound are transmitted into tissue (e.g., by using an ultrasound probe), sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound probes, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a method includes providing, by a first processing device in operative communication with an ultrasound device, an instruction to collect sets of ultrasound data from multiple positions of the ultrasound device; receiving, from the ultrasound device, the sets of ultrasound data; transmitting the sets of ultrasound data, or portions or indications thereof, to a second processing device; receiving, from the second processing device, an indication of a selected set of ultrasound data; providing an instruction to move the ultrasound device to a position at which the selected set of ultrasound data was collected; and receiving further ultrasound data from the ultrasound device at the position at which the selected set of ultrasound data was collected.

In some embodiments, providing the instruction to collect the sets of ultrasound data from the multiple positions of the ultrasound device includes providing an instruction to collect sets of ultrasound data from multiple locations of the ultrasound device, each of the sets of ultrasound data includes ultrasound data collected at a particular location of the ultrasound device, and providing the instruction to move the ultrasound device to the position at which the selected set of ultrasound data was collected includes providing an instruction to translate the ultrasound device to a location of the ultrasound device at which the selected set of ultrasound data was collected. In some embodiments, providing the instruction to collect the sets of ultrasound data from the multiple locations of the ultrasound device includes providing an instruction to move the ultrasound device across substantially all of an anatomical area. In some embodiments, the anatomical area is greater than 25 cm² in area. In some embodiments, providing the instruction to collect the sets of ultrasound data from the multiple locations of the ultrasound device includes providing an instruction to move the ultrasound device in a serpentine path. In some embodiments, providing the instruction to collect the sets of ultrasound data from the multiple locations of the ultrasound device includes providing an instruction to move the ultrasound device in a spiral path. In some embodiments, providing the instruction to collect the sets of ultrasound data from the multiple locations of the ultrasound device includes providing an instruction to maintain the ultrasound device at its current rotation and/or its current tilt while translating the ultrasound device. In some embodiments, providing the instruction to translate the ultrasound device to the location of the ultrasound device at which the selected set of ultrasound data was collected includes providing an instruction to maintain the ultrasound device at its current rotation and/or its current tilt while translating the ultrasound device.

In some embodiments, providing the instruction to collect the sets of ultrasound data from the multiple positions of the ultrasound device includes providing an instruction to collect sets of ultrasound data from multiple rotations of the ultrasound device, each of the sets of ultrasound data includes ultrasound data collected at a particular rotation of the ultrasound device, and providing the instruction to move the ultrasound device to the position at which the selected set of ultrasound data was collected includes providing an instruction to rotate the ultrasound device to a rotation of the ultrasound device at which the selected set of ultrasound data was collected. In some embodiments, providing the instruction to collect the sets of ultrasound data from the multiple rotations of the ultrasound device includes providing an instruction to rotate the ultrasound device between approximately 85 degrees and 95 degrees about a location. In some embodiments, providing the instruction to collect the sets of ultrasound data from the multiple rotations of the ultrasound device includes providing an instruction to rotate the ultrasound device between approximately 175 degrees and 185 degrees about a location. In some embodiments, providing the instruction to collect the sets of ultrasound data from the multiple rotations of the ultrasound device includes providing an instruction to rotate the ultrasound device between approximately 355 degrees and 365 degrees about a location. In some embodiments, providing the instruction to collect the sets of ultrasound data from the multiple rotations of the ultrasound device includes providing an instruction to maintain the ultrasound device at its current location and/or its current tilt while rotating the ultrasound device. In some embodiments, providing the instruction to rotate the ultrasound device to the rotation of the ultrasound device at which the selected set of ultrasound data was collected includes providing an instruction to maintain the ultrasound device at its current location and/or its current tilt while rotating the ultrasound device.

In some embodiments, providing the instruction to collect the sets of ultrasound data from the multiple positions of the ultrasound device includes providing an instruction to collect sets of ultrasound data from multiple tilts of the ultrasound device, each of the sets of ultrasound data includes ultrasound data collected at a particular tilt of the ultrasound device, and providing the instruction to move the ultrasound device to the position at which the selected set of ultrasound data was collected includes providing an instruction to move the ultrasound device to a tilt of the ultrasound device at which the selected set of ultrasound data was collected. In some embodiments, providing the instruction to collect the sets of ultrasound data from the multiple tilts of the ultrasound device includes providing an instruction to tilt the ultrasound device between approximately 85 degrees and 95 degrees about a location. In some embodiments, providing the instruction to collect the sets of ultrasound data from the multiple tilts of the ultrasound device includes providing an instruction to tilt the ultrasound device approximately 180 degrees about a location. In some embodiments, providing the instruction to collect the sets of ultrasound data from the multiple tilts of the ultrasound device includes providing an instruction to maintain the ultrasound device at its current location and/or its current rotation while tilting the ultrasound device. In some embodiments, providing the instruction to tilt the ultrasound device to the tilt of the ultrasound device at which the selected set of ultrasound data was collected includes providing an instruction to maintain the ultrasound device at its current location and/or its current rotating while tilting the ultrasound device.

In some embodiments, the method further includes receiving the instruction to collect the sets of ultrasound data from the multiple positions of the ultrasound device from the second processing device.

Some aspects include at least one non-transitory computer-readable storage medium storing processor-executable instructions that, when executed by at least one processor, cause the at least one processor to perform the above aspects and embodiments. Some aspects include an ultrasound system having a processing device configured to perform the above aspects and embodiments.

Some aspects include at least one non-transitory computer-readable storage medium storing processor-executable instructions that, when executed by at least one processor, cause the at least one processor to perform the above aspects and embodiments. Some aspects include an ultrasound system having a processing device configured to perform the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates a schematic block diagram of an example ultrasound system, in accordance with certain embodiments described herein;

FIG. 2 illustrates an example perspective view of the ultrasound device, in accordance with certain embodiments described herein;

FIGS. 3-6 illustrate an example of moving an ultrasound device to a target position on a subject, in accordance with certain embodiments described herein. FIG. 3 shows the ultrasound device at a starting position. FIG. 4 shows a position of the ultrasound device after it has been translated to a target location on the subject. FIG. 5 shows a position of the ultrasound device after it has been rotated to a target rotation while remaining at the target location. FIG. 6 shows a position of the ultrasound device after it has been tilted to a target tilt while remaining at the target location and target rotation;

FIG. 7 illustrates an example process for collection of ultrasound data, in accordance with certain embodiments described herein;

FIG. 8 illustrates an example instruction that may be provided by a processing device, in accordance with certain embodiments described herein;

FIG. 9 illustrates another example instruction that may be provided by the processing device, in accordance with certain embodiments described herein;

FIG. 10 illustrates another example instruction that may be provided by the processing device, in accordance with certain embodiments described herein;

FIG. 11 illustrates another example instruction that may be provided by the processing device, in accordance with certain embodiments described herein;

FIG. 12 illustrates another example instruction that may be provided by a processing device, in accordance with certain embodiments described herein;

FIG. 13 illustrates another example instruction that may be provided by a processing device, in accordance with certain embodiments described herein;

FIG. 14 illustrates another example instruction that may be provided by a processing device, in accordance with certain embodiments described herein;

FIG. 15 illustrates an example instruction that may be provided by a processing device, in accordance with certain embodiments described herein;

FIG. 16 illustrates another example instruction that may be provided by a processing device, in accordance with certain embodiments described herein;

FIG. 17 illustrates another example instruction that may be provided by a processing device, in accordance with certain embodiments described herein;

FIG. 18 illustrates another example instruction that may be provided by a processing device, in accordance with certain embodiments described herein;

FIG. 19 illustrates an example instruction that may be provided by a processing device, in accordance with certain embodiments described herein;

FIG. 20 illustrates another example instruction that may be provided by a processing device, in accordance with certain embodiments described herein;

FIG. 21 illustrates another example instruction that may be provided by a processing device, in accordance with certain embodiments described herein;

FIG. 22 illustrates another example instruction that may be provided by a processing device, in accordance with certain embodiments described herein;

FIG. 23 illustrates another example instruction that may be provided by a processing device, in accordance with certain embodiments described herein; and

FIG. 24 illustrates another example instruction that may be provided by a processing device, in accordance with certain embodiments described herein.

### DETAILED DESCRIPTION

Conventional ultrasound systems are large, complex, and expensive systems that are typically only purchased by large medical facilities with significant financial resources. Recently, cheaper and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Pat. Pub. No. US-2017-0360397-A1, which is incorporated by reference herein in its entirety. The reduced cost and increased portability of these new ultrasound devices may make them significantly more accessible to the general public than conventional ultrasound devices.

The inventors have recognized and appreciated that although the reduced cost and increased portability of ultrasound imaging devices makes them more accessible to the general populace, people who could make use of such devices have little to no training for how to use them. Ultrasound examinations often include the acquisition of ultrasound images that contain a view of a particular anatomical structure (e.g., an organ) of a subject. Acquisition of these ultrasound images typically requires considerable skill. For example, an ultrasound technician operating an ultrasound device may need to know where the anatomical structure to be imaged is located on the subject and further how to properly position the ultrasound device on the subject to capture a medically relevant ultrasound image of the anatomical structure. Holding the ultrasound device a little too high or a little too low on the subject may make the difference between capturing a medically relevant ultrasound image and capturing a medically irrelevant ultrasound image. As a result, non-expert operators of an ultrasound device may have considerable trouble capturing medically relevant ultrasound images of a subject. Common mistakes by these non-expert operators include capturing ultrasound images of the incorrect anatomical structure and capturing foreshortened (or truncated) ultrasound images of the correct anatomical structure.

For example, a small clinic without a trained ultrasound technician on staff may purchase an ultrasound device to help diagnose patients. In this example, a nurse at the small clinic may be familiar with ultrasound technology and human physiology, but may know neither which anatomical views of a patient need to be imaged in order to identify medically-relevant information about the patient nor how to obtain such anatomical views using the ultrasound device. In another example, an ultrasound device may be issued to a patient by a physician for at-home use to monitor the patient's heart. In all likelihood, the patient understands neither human physiology nor how to image his or her own heart with the ultrasound device.

Accordingly, the inventors have developed assistive ultrasound imaging technology for instructing an operator of an ultrasound device how to move the ultrasound device relative to an anatomical area of a subject in order to capture a medically relevant ultrasound image. Providing instructions to the operator for positioning the ultrasound device in order to collect ultrasound data capable of being transformed into an ultrasound image containing a target anatomical view (for simplicity, referred to herein as “target ultrasound data”) may be difficult. For example, if the target ultrasound data can be collected by placing the ultrasound device at a specific position relative to a subject (where position includes location, rotation, and tilt of the ultrasound device), one option for instructing the operator to collect the target ultrasound data may be to provide an explicit description of the target position and instructing the operator to place the ultrasound device at the target position. However, this may be difficult if there is not an easy way to describe the target position, either visually or with words.

Some embodiments include techniques that may enable the operator to collect, with the ultrasound device, the target ultrasound data without providing an explicit description of the target position or an identification of the target position as such. In these embodiments, the operator may be provided with a description of a path that does not explicitly mention the target position, but which includes the target position, as well as other locations (for simplicity, referred to herein as “non-target positions”) where ultrasound data not capable of being transformed into an ultrasound image of the target anatomical view (for simplicity, referred to herein as “non-target ultrasound data”) is collected. The path may relate to one or more of location, rotation, and tilt. Moving the ultrasound device along the path should, if done correctly, result in collection of the target ultrasound data. While moving the ultrasound device along such a path causes the ultrasound device to collect non-target ultrasound data in addition to the target ultrasound data, the inventors have recognized that describing such a path may be easier than describing the target position. Furthermore, because the description of such a path may be less complex than the description of the target position, following instructions to move the ultrasound device along such a path may be easier for an operator than following instructions to place the ultrasound device at the target position. For example, consider an ultrasound device that has been placed in a target location and rotation, but needs to be moved to a target tilt in order to collect a target anatomical view. Instructing the operator to move the ultrasound device along a path that involves tilting the ultrasound device through approximately 180 degrees about a particular anatomical location may be easier than instructing the operator to tilt the ultrasound device to a particular angle within the 180-degree arc relative to the anatomical location. The inventors have therefore recognized that it may be beneficial to instruct the operator to move the ultrasound device along a path whereby the ultrasound device collects target and non-target ultrasound data, as such an instruction may be easier to describe and follow than a specific description of the target position. In other words, purposefully instructing the operator to collect non-target ultrasound data may, unexpectedly and non-intuitively, help the operator to collect the target ultrasound data.

It may be desirable for an operator to collect ultrasound data with the ultrasound device at a particular position for a specified period of time. For example, an ultrasound device at a particular position may collect a series of ultrasound images depicting a target anatomical view of the heart proceeding through multiple heart cycles. The inventors have recognized that a user may first move an ultrasound device along a path, such as tilting the ultrasound device through 180 degrees about a particular anatomical location, while data that includes ultrasound data is collected by the ultrasound device at various tilts along the path. A remote expert may receive the ultrasound data, select a particular set of ultrasound data that was collected at a target tilt, and based on the data collected at the same tilt as the selected ultrasound data, the user may be instructed to move the ultrasound device back to the target tilt to collect more ultrasound data. For example, the user may be instructed to move the ultrasound device back to the target tilt based on motion and/or orientation data from the ultrasound device.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

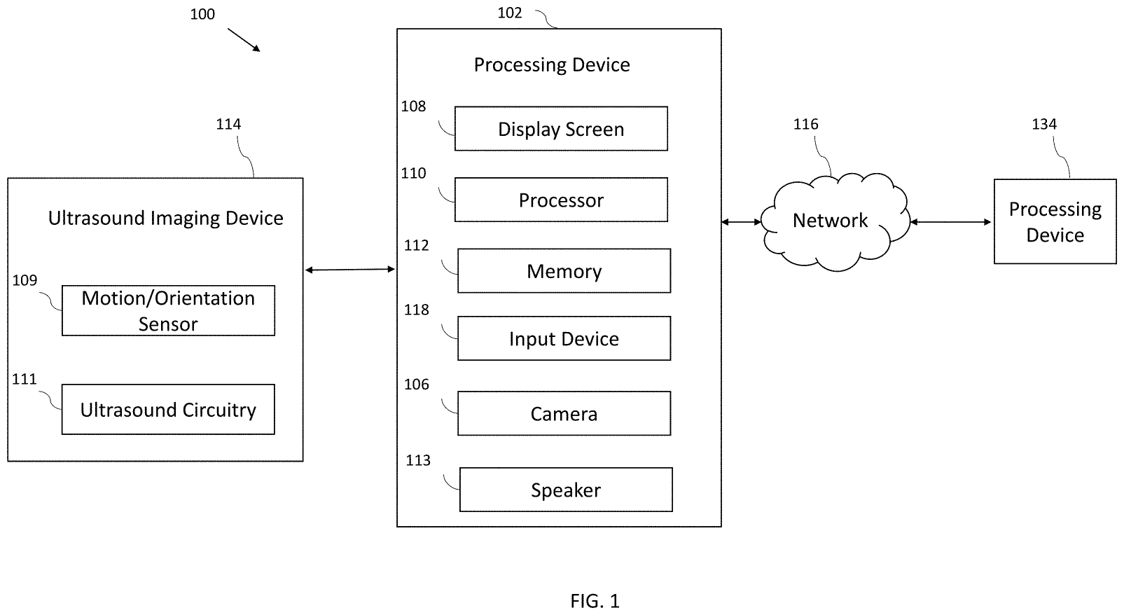

FIG. 1 illustrates a schematic block diagram of an example ultrasound system 100, in accordance with certain embodiments described herein. The ultrasound system 100 includes an ultrasound device 114, a processing device 102, a network 116, and a processing device 134.

The ultrasound device 114 includes a motion and/or orientation sensor 109 and ultrasound circuitry 111. The processing device 102 includes a camera 106, a display screen 108, a processor 110, memory 112, an input device 118, and a speaker 113. The processing device 102 is in wired (e.g., through a lightning connector or a mini-USB connector) and/or wireless communication (e.g., using BLUETOOTH, ZIGBEE, and/or WiFi wireless protocols) with the ultrasound device 114. The processing device 102 is in wireless communication with the processing device 134 over the network 116.

The ultrasound device 114 may be configured to generate ultrasound data that may be employed to generate an ultrasound image. The ultrasound device 114 may be constructed in any of a variety of ways. In some embodiments, the ultrasound device 114 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 111 may be configured to generate the ultrasound data. The ultrasound circuitry 111 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed on the same chip as other electronic components in the ultrasound circuitry 111 (e.g., transmit circuitry, receive circuitry, control circuitry, power management circuitry, and processing circuitry) to form a monolithic ultrasound device. The ultrasound device 114 may transmit ultrasound data and/or ultrasound images to the processing device 102 over a wired (e.g., through a lightning connector or a mini-USB connector) and/or wireless (e.g., using BLUETOOTH, ZIGBEE, and/or WiFi wireless protocols) communication link.

The motion and/or orientation sensor 109 may be configured to generate motion and/or orientation data regarding the ultrasound device 114. For example, the motion and/or orientation sensor 109 may be configured to generate data regarding acceleration of the ultrasound device 114, data regarding angular velocity of the ultrasound device 114, and/or data regarding magnetic force acting on the ultrasound device 114 (which, due to the magnetic field of the earth, may be indicative of orientation relative to the earth). The motion and/or orientation sensor 109 may include an accelerometer, a gyroscope, and/or a magnetometer. Depending on the sensors present in the motion and/or orientation sensor 109, the motion and/or orientation data generated by the motion and/or orientation sensor 109 may describe three degrees of freedom, six degrees of freedom, or nine degrees of freedom for the ultrasound device 114. For example, the motion and/or orientation sensor may include an accelerometer, a gyroscope, and/or magnetometer. Each of these types of sensors may describe three degrees of freedom. If the motion and/or orientation sensor includes one of these sensors, the motion and/or orientation sensor may describe three degrees of freedom. If the motion and/or orientation sensor includes two of these sensors, the motion and/or orientation sensor may describe two degrees of freedom. If the motion and/or orientation sensor includes three of these sensors, the motion and/or orientation sensor may describe nine degrees of freedom. The ultrasound device 114 may transmit motion and/or orientation data to the processing device 102 over a wired (e.g., through a lightning connector or a mini-USB connector) and/or wireless (e.g., using BLUETOOTH, ZIGBEE, and/or WiFi wireless protocols) communication link.

Referring now to the processing device 102, the processor 110 may include specially-programmed and/or special-purpose hardware such as an application-specific integrated circuit (ASIC). For example, the processor 110 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 102 may be configured to process the ultrasound data received from the ultrasound device 114 to generate ultrasound images for display on the display screen 108. The processing may be performed by, for example, the processor 110. The processor 110 may also be adapted to control the acquisition of ultrasound data with the ultrasound device 114. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, or at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 102 may be configured to perform certain of the processes described herein using the processor 110 (e.g., one or more computer hardware processors) and one or more articles of manufacture that include non-transitory computer-readable storage media such as the memory 112. The processor 110 may control writing data to and reading data from the memory 112 in any suitable manner. To perform certain of the processes described herein, the processor 110 may execute one or more processor-executable instructions stored in one or more non-transitory computer-readable storage media (e.g., the memory 112), which may serve as non-transitory computer-readable storage media storing processor-executable instructions for execution by the processor 110. The camera 106 may be configured to detect light (e.g., visible light) to form an image (which may be a frame of a video). The display screen 108 may be configured to display images and/or videos, and may be, for example, a liquid crystal display (LCD), a plasma display, and/or an organic light emitting diode (OLED) display on the processing device 102. The input device 118 may include one or more devices capable of receiving input from a user and transmitting the input to the processor 110. For example, the input device 118 may include a keyboard, a mouse, a microphone, touch-enabled sensors on the display screen 108, and/or a microphone. The speaker 113 may be configured to output audio from the processing device 102. The display screen 108, the input device 118, the camera 106, and the speaker 113 may be communicatively coupled to the processor 110 and/or under the control of the processor 110.

It should be appreciated that the processing device 102 may be implemented in any of a variety of ways. For example, the processing device 102 may be implemented as a handheld device such as a mobile smartphone or a tablet. Thereby, a user of the ultrasound device 114 may be able to operate the ultrasound device 114 with one hand and hold the processing device 102 with another hand. In other examples, the processing device 102 may be implemented as a portable device that is not a handheld device, such as a laptop. In yet other examples, the processing device 102 may be implemented as a stationary device such as a desktop computer. The processing device 102 may be connected to the network 116 over a wired connection (e.g., via an Ethernet cable) and/or a wireless connection (e.g., over a WiFi network). The processing device 102 may thereby communicate with (e.g., transmit data to) the processing device 134 over the network 116. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application). FIG. 1 should be understood to be non-limiting. For example, the ultrasound system 100 may include fewer or more components than shown and the processing device 102 may include fewer or more components than shown.

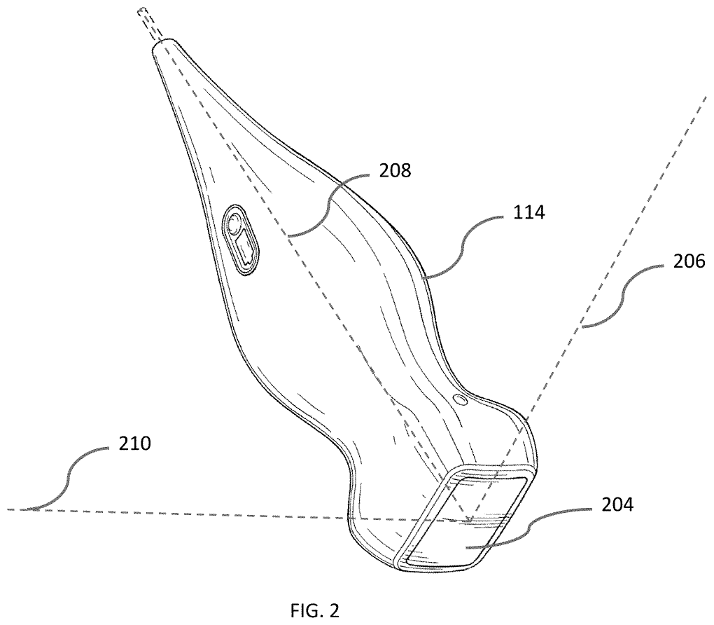

FIG. 2 illustrates an example perspective view of the ultrasound device 114, in accordance with certain embodiments described herein. The ultrasound device 114 includes a sensor 204, a roll axis 208, a pitch axis 206, and a yaw axis 210 of the ultrasound device 114. An orientation of the ultrasound device 114 may be defined by rotation angles about these axes, where roll refers to rotation angle about the roll axis 208, pitch refers to rotation angle about the pitch axis 206, and yaw refers to rotation angle about the yaw axis 210. A particular rotation angle about the roll axis 208 may be referred to as a rotation of the ultrasound device 114, a particular rotation angle about the pitch axis 206 may be referred to as a tilt of the ultrasound device 114, and a particular rotation angle about the yaw axis 210 may be referred to as a rock of the ultrasound device 114.

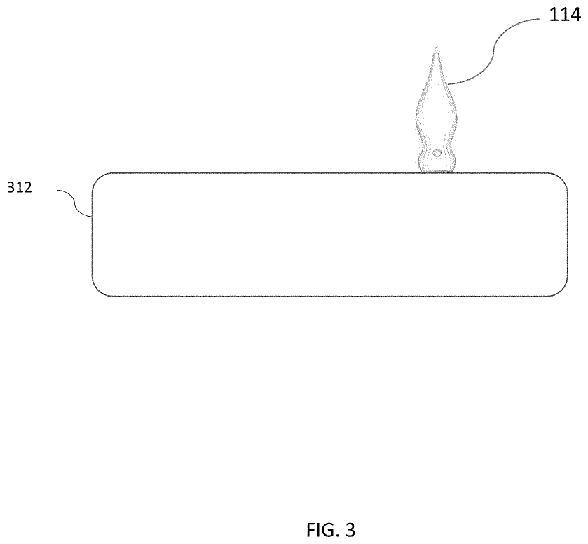

FIGS. 3-6 illustrate an example of moving an ultrasound device to a target position on a subject 312 (shown from a side view), in accordance with certain embodiments described herein. A position of the ultrasound device 114 may refer to a particular location, rotation, and tilt of the ultrasound device 114. The target position may be a position in which the ultrasound device 114 can collect a target anatomical view from the subject 312. The ultrasound device 114 may be in the target position when the ultrasound device 114 is at a particular target location, rotation, and tilt on the subject 312.

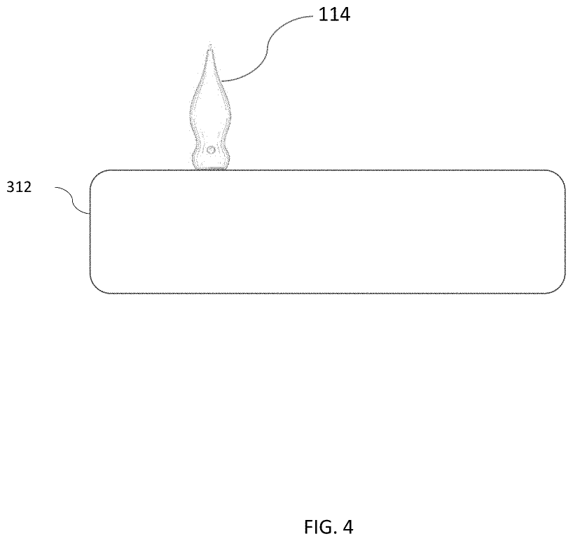

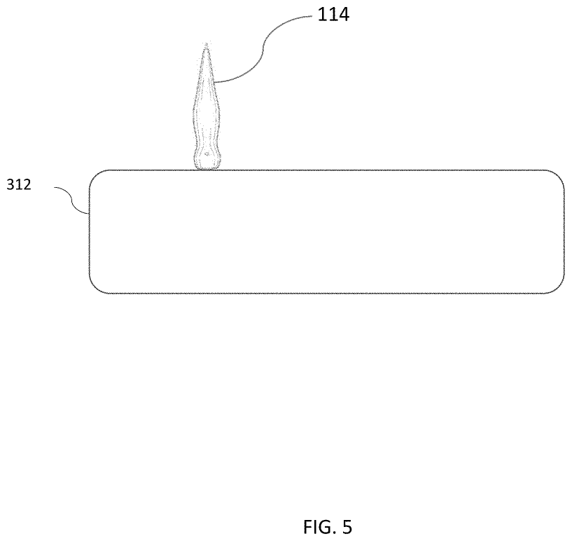

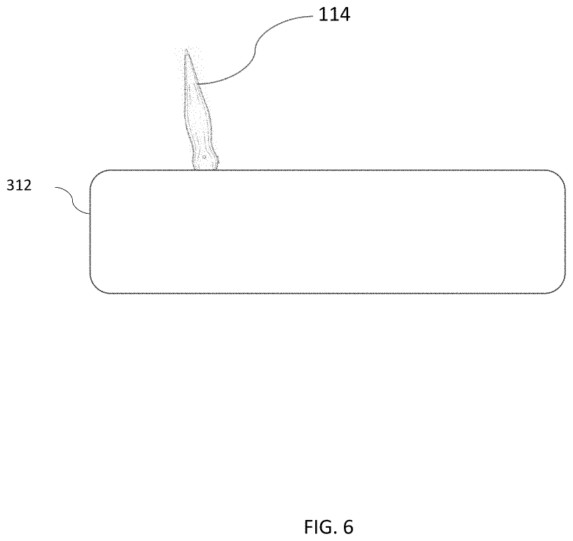

FIG. 3 shows the ultrasound device 114 at a starting position. FIG. 4 shows a position of the ultrasound device 114 after it has been translated to a target location on the subject 312. FIG. 5 shows a position of the ultrasound device 114 after it has been rotated to a target rotation while remaining at the target location. FIG. 6 shows a position of the ultrasound device 114 after it has been tilted to a target tilt while remaining at the target location and target rotation. When the ultrasound device 114 is at the target location, target rotation, and target tilt, the ultrasound device may be in the target position where the ultrasound device 114 can collect the target anatomical view from the subject 312.

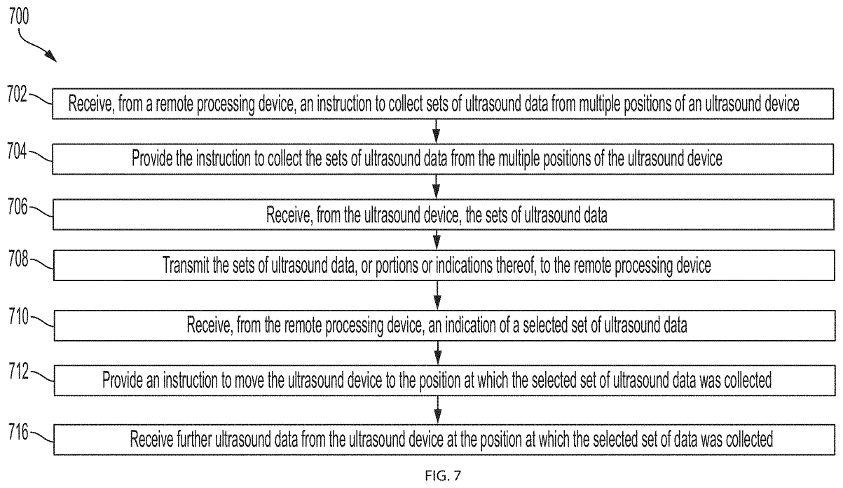

FIG. 7 illustrates an example process 700 for collection of ultrasound data, in accordance with certain embodiments described herein. The process 700 is performed by a local processing device (e.g., the processing device 102) in operative communication with an ultrasound device (e.g., the ultrasound device 114). The local processing device may be local to the ultrasound device and/or a user of the ultrasound device. The local processing device may be in communication with a remote processing device (e.g., the processing device 134) that may be local to a remote entity (e.g., a remote expert, medical professional, or other user) but remote from the user. The local processing device and the remote processing device may be in communication over a wired communication link (e.g., over Ethernet, a Universal Serial Bus (USB) cable, or a Lightning cable) or over a wireless communication link (e.g., over a BLUETOOTH, WiFi, or ZIGBEE wireless communication link). It should be appreciated that the process 700 may be performed by other devices, such as the ultrasound device itself. The process 700 may include instructing a user to attain a target position of an ultrasound device by providing instructions to a user to move the ultrasound device to multiple positions.

In act 702, the local processing device receives, from the remote processing device, an instruction to collect sets of ultrasound data from multiple positions of an ultrasound device on a subject. The ultrasound data may include, for example, raw acoustical data, scan lines generated from raw acoustical data, or ultrasound images generated from raw acoustical data. In some embodiments, the multiple positions may include multiple locations of the ultrasound device on the subject. In some embodiments, the multiple positions may include multiple rotations of the ultrasound device on the subject. In some embodiments, the multiple positions may include multiple tilts of the ultrasound device on the subject. In some embodiments, each of the multiple positions may include multiple locations, multiple rotations, and/or multiple tilts.

In some embodiments, the instruction may be to translate the ultrasound device in a serpentine or spiral fashion across substantially all of an anatomical area (e.g., the cardiac region, the torso, the abdomen, etc.) or a portion of an anatomical area (e.g., the upper left portion of the torso). In some embodiments, the instruction may be to translate the ultrasound device across an anatomical area that is greater than, for example, 5 cm² in area, 10 cm² in area, 15 cm² in area, 20 cm² in area, 25 cm² in area, 30 cm² in area, 35 cm² in area, 40 cm² in area, 45 cm² in area, 50 cm² in area, or any other suitable size. In some embodiments, the instruction may include instructions to maintain the ultrasound device at its current rotation and/or its current tilt while translating the ultrasound device.

In some embodiments, the instruction may be to rotate the ultrasound device. In some embodiments, the instruction may be to rotate the ultrasound device 360 degrees, 270 degrees, 180 degrees, 90 degrees, between approximately 85 degrees and 95 degrees, between approximately 175 degrees and 185 degrees, between approximately 265 degrees and 275 degrees, between approximately 355 degrees and 365 degrees, or any suitable number of degrees (including any value or range of values within the listed ranges), about the anatomical location, while collecting ultrasound data with the ultrasound device at various rotations. The instruction may include instructions to maintain the ultrasound device at its current location and/or its current tilt while rotating the ultrasound device.

In some embodiments, the instruction may be to tilt the ultrasound device. In some embodiments, the instruction may be to tilt the ultrasound device through 180 degrees, 150 degrees, 120 degrees, 90 degrees, 60 degrees, 30 degrees, any value within 10% of any of those values listed, any value within 20% of those values listed, or any suitable number of degrees, about a location, while collecting ultrasound data with the ultrasound device at various tilts. The instruction may include instructions to maintain the ultrasound device at its current location and/or its current rotation while tilting the ultrasound device.

In some embodiments, the instruction may include an image or a video. For example, the remote expert may select a predefined image or video from a display on the remote processing device and the remote processing device may transmit the selection to the local processing device. As another example, the remote expert may perform an action (e.g., demonstrate a movement with a real or mock ultrasound device) that is captured by a camera on the remote processing device as a video signal, and the remote processing device may transmit the video signal to the local processing device. In some embodiments, the instruction may include words. For example, the remote expert may select predefined words from a display on the remote processing device and the remote processing device may transmit the selection to the local processing device. As another example, the remote expert may speak words that are captured by a microphone on the remote processing device as an audio signal and the remote processing device may transmit the audio signal to the local processing device.

As another example, the remote processing device may receive a video from the local processing device that depicts the current position of the ultrasound device on the subject. The video may be captured by a camera on the local processing device. For example, the user may hold the processing device in one hand and hold the ultrasound device in view of the camera on the local processing device with the other hand. The remote processing device may further show multiple directions for moving the ultrasound device relative to the subject, and these directions may be superimposed on the video of the subject. For example, the multiple directions may be shown as multiple arrows indicating directions for translating, rotating, and/or tilting the ultrasound device. The remote expert may select one of the directions, and the remote processing device may transmit an indication of the selected direction to the local processing device. The local processing device may then display, as the instruction for moving the ultrasound device, the selected direction superimposed on the video of the subject. The displays of the directions superimposed on the video of the subject may be considered an augmented reality interface.

In some embodiments, rather than receiving the instruction from a remote processing device, the local processing may generate the instruction automatically. For example, if a user selects from the local processing device a particular anatomical feature to be imaged or a particular imaging protocol to be used, the local processing device may retrieve from a database a predetermined instruction associated with the particular anatomical feature to be imaged or the particular imaging protocol. Thus, in some embodiments, act 702 may be absent. The process 700 proceeds from act 702 to act 704.

In act 704, the local processing device provides the instruction received in act 702. Depending on the type and content of the instruction, the local processing device may display an image, video, or words on a display screen of the local processing device and/or output words from a speaker of the local processing device in order to provide the instruction. The process 700 proceeds from act 704 to act 706.

In act 706, the local processing device receives, from the ultrasound device, sets of ultrasound data. For example, the user may have moved the ultrasound device to multiple positions based on the instruction provided at act 704 and collected ultrasound data at the multiple positions. The ultrasound data may include, for example, raw acoustical data, scan lines generated from raw acoustical data, or ultrasound images generated from raw acoustical data. In some embodiments, the ultrasound device may generate scan lines and/or ultrasound images from raw acoustical data and transmit the scan lines and/or ultrasound images to the local processing device. In other embodiments, the ultrasound device may transmit the raw acoustical data to the local processing device and the local processing device may generate the scan lines and/or ultrasound images from the raw acoustical data. In still other embodiments, the ultrasound device may generate scan lines from the raw acoustical data, transmit the scan lines to the local processing device, and the local processing device may generate ultrasound images from the scan lines.

In some embodiments, the local processing device may generate pose data. The pose data may be generated based on data regarding the location and orientation of the ultrasound device relative to the local processing device when the ultrasound device collected the sets of ultrasound data. The local processing device may generate the pose data by collecting motion and/or orientation data from the local processing device, video data from the local processing device, and/or motion and/or orientation data from the ultrasound device. In some embodiments, the local processing device may determine, based on video collected by the local processing device that depicts the ultrasound device, a translation of the ultrasound device relative to the local processing device. The video may be collected by a camera on the local processing device. In some embodiments, a user may hold the ultrasound device in one hand and hold the local processing device in the other hand such that the ultrasound device is in view of the camera on the local processing device. In some embodiments, a user may hold the ultrasound device in one hand and a holder (e.g., a stand having a clamp for holding the local processing device) may hold the local processing device such that the ultrasound device is in view of the camera on the local processing device.

In some embodiments, a statistical model may be trained to determine the translation of the ultrasound device relative to the local processing device. In some embodiments, the statistical model may be trained as a keypoint localization model with training input and output data. Multiple images of the ultrasound device may be inputted to the statistical model as training input data. As training output data, an array of values that is the same size as the inputted image may be inputted to the statistical model, where the pixel corresponding to the location of the tip of the ultrasound device (namely, the end of the ultrasound device opposite the sensor portion) in the image is manually set to a value of 1 and every other pixel has a value of 0 (although the values 1 and 0 are just examples values, and other values may be used. Based on this training data, the statistical model may learn to output, based on an inputted image (e.g., a frame of the video of the ultrasound device captured by the local processing device), an array of values that is the same size as the inputted image, where each pixel in the array consists of a probability that that pixel is where the tip of the ultrasound image is located in the inputted image. The local processing device may then predict that the pixel having the highest probability represents the location of the tip of the ultrasound image and output the horizontal and vertical coordinates of this pixel.

In some embodiments, a statistical model may be trained to use regression to determine the translation of the ultrasound device relative to the local processing device. Multiple images of the ultrasound device may be inputted to the statistical model as training input data. As training output data, each input image may be manually labeled with two numbers, namely the horizontal and vertical pixel coordinates of the tip of the ultrasound device (namely, the end of the ultrasound device opposite the sensor portion) in the image. Based on this training data, the statistical model may learn to output, based on an inputted image (e.g., a frame of the video of the ultrasound device captured by the local processing device), the horizontal and vertical pixel coordinates of the tip of the ultrasound device in the image.

In some embodiments, a statistical model may be trained as a segmentation model to determine the translation of the ultrasound device relative to the local processing device. Multiple images of the ultrasound device may be inputted to the statistical model as training input data. As training output data, a segmentation mask may be inputted to the statistical model, where the segmentation mask is an array of values equal in size to the image, and pixels corresponding to locations within the ultrasound device in the image are manually set to 1 and other pixels are set to 0. Based on this training data, the statistical model may learn to output, based on an inputted image (e.g., a frame of the video of the ultrasound device captured by the local processing device), a segmentation mask where each pixel has a value representing the probability that the pixel corresponds to a location within the ultrasound device in the image (values closer to 1) or outside the ultrasound device (values closer to 0). Horizontal and vertical pixel coordinates representing a single location of the ultrasound device in the image may then be derived (e.g., using averaging or some other method for deriving a single value from multiple values) from this segmentation mask.

In some embodiments, to determine the depth (z-direction) of the tip of the ultrasound device relative to the local processing device, the local processing device may use a depth camera on the local processing device. For example, the depth camera may use disparity maps or structure light cameras. Such cameras may be considered stereo cameras in that they may use two cameras at different locations on the local processing device that simultaneously capture two images, and the disparity between the two images may be used to determine the depth of the tip of the ultrasound device depicted in both images. In some embodiments, a time-of-flight camera may be used to determine the depth of the tip of the ultrasound device. In some embodiments, the local processing device may use such depth cameras to determine the depth of the tip of the ultrasound device, and use a statistical model to determine horizontal and vertical coordinates of the tip of the ultrasound device in video captured with just one camera, as described above. However, in other embodiments, a statistical model may be trained to determine the depth from the image captured with just one camera. To train the statistical model, multiple images may be labeled with the depth of the tip of the ultrasound device in each image, where the depth may be determined manually or determined using any other method such as a depth camera. Thus, the local processing device may use a statistical model to determine horizontal and vertical coordinates of the tip of the ultrasound device as well as the depth of the tip based on video captured with just one camera. In some embodiments, the local processing device may assume a predefined depth as the depth of the tip of the ultrasound device relative to the local processing device.

Using camera intrinsics (e.g., focal lengths, skew coefficient, and principal points), the local processing device may convert the horizontal and vertical pixel coordinates of the tip of the ultrasound device into the horizontal (x-direction) and vertical (y-direction) distance of the tip of the ultrasound device relative to the local processing device (more precisely, relative to the camera of the local processing device). Note that the local processing device may also use the depth to determine the horizontal and vertical distance. The distances of the tip of the ultrasound device relative to the local processing device in the x-, y-, and z-directions may be considered the translation of the tip of the ultrasound device relative to the local processing device. It should be appreciated that as an alternative to the tip of the ultrasound device, any feature on the ultrasound device may be used instead.

In some embodiments, an auxiliary marker on the ultrasound device may be used to determine the distances of that feature relative to the local processing device in the x-, y-, and z-directions based on video of the ultrasound device captured by the local processing device, using pose estimation techniques and without using statistical models. For example, the auxiliary marker may be a marker conforming to the ArUco library, a color band, or some feature that is part of the ultrasound device itself.

In some embodiments, the local processing device may determine, based on motion and/or orientation data from the local processing device and motion and/or orientation data from the ultrasound device, an orientation of the ultrasound device relative to the local processing device. The motion and/or orientation data from each device may describe acceleration of the device, angular velocity of the device, and/or the magnetic field in the vicinity of the device. The motion and/or orientation data may be generated by an accelerometer, a gyroscope, and/or magnetometer, which together may constitute an inertial measurement unit (IMU). Using sensor fusion techniques (e.g., based on Kalman filters, complimentary filters, and/or algorithms such as the Madgwick algorithm), this motion and/or orientation data may be used to generate the roll, pitch, and yaw angles of the device relative to a coordinate system defined by the directions of the local gravitational acceleration and the local magnetic field. If the roll, pitch, and yaw angles of each device are described by a rotation matrix, then multiplying the rotation matrix of the local processing device by the inverse of the rotation matrix of the ultrasound device may produce a matrix describing the orientation (namely, the roll, pitch, and yaw angles) of the ultrasound device relative to the local processing device.

In some embodiments, other methods may be used to determine the orientation of the ultrasound device relative to the local processing device. For example, a statistical model may be trained to locate a set of different features of the ultrasound device in the video of the ultrasound device captured by the local processing device (e.g., using methods described above for locating the tip of the ultrasound device in an image), from which the orientation of the ultrasound device may be uniquely determined. In some embodiments, a statistical model may be trained to determine, from an image or video of the ultrasound device captured by the local processing device, the orientation of the ultrasound device relative to the local processing device using regression. The statistical model may be trained on training input and output data, where the training input data is an image of the ultrasound device captured by the local processing device and the output data consists of three numbers, namely the roll, pitch, and yaw angles (in other words, the orientation) of the ultrasound device relative to the local processing device. The roll, pitch, and yaw angles for the output data may be determined from the sensor on the ultrasound device and the sensor on the local processing device using the method described above. In some embodiments, the orientation of the ultrasound device relative to the earth may be determined up to the angle of the ultrasound device around the axis of gravity based on motion and/or orientation sensors on the ultrasound device (e.g., based on the accelerometer and/or gyroscope), and the orientation of the ultrasound device around the axis of gravity may be determined from video of the ultrasound device captured by the local processing device (rather than, for example, a magnetometer of the ultrasound device) using a statistical model. The statistical model may be trained on images labeled with the angle around the axis of gravity, where the label is derived from magnetometer data. In some embodiments, methods described for determining orientation using the video of the ultrasound device and using motion and/or orientation sensors may both be used and combined into a single prediction that may be more reliable than if only one method were used.

The location and orientation of the ultrasound device relative to the local processing device may together constitute a pose data for the ultrasound device relative to the local processing device. It should be appreciated that other methods for determining the pose of the ultrasound device relative to the local processing device may be used.

As will be discussed below, in act 712, the local processing device provides an instruction for moving the ultrasound device to the position at which the selected set of ultrasound data was collected (this position may be referred to as the target pose). In some embodiments, in act 712, the local processing device may determine the current pose of the ultrasound device, compare the current pose to the target pose, and provide an instruction for bringing the current pose closer to the target pose. It may be helpful to determine the target pose of the local processing device relative to the subject (rather than relative to the local processing device) in case the local processing device moves between act 706 and 712. If the local processing device moves between act 706 and 712, a target pose of the ultrasound device relative to the local processing device may not necessarily be in the same position on the subject at act 706 and act 712. Thus, in some embodiments, the local processing device may track the pose of the local processing device relative to the external world (e.g., using an augmented reality toolkit such as ARkit available on the local processing device). Based on this pose, the current pose of the local processing device relative to the ultrasound device, and an assumption that the subject being imaged does not move relative to the external world, the local processing device may determine the current and target poses of the ultrasound device relative to the subject being imaged. Alternatively or additionally, the local processing device may be stationary (e.g., the local processing device may be held by a holder such as a clamp) or the local processing device may instruct the user to hold the local processing device stationary. This may obviate a need to track the pose of the local processing device relative to the external world, such that determining the pose of the ultrasound device relative to the local processing device may be sufficient.

In some embodiments, a statistical model may be trained to determine, based on ultrasound data, a location and orientation of the ultrasound device relative to the subject when the ultrasound device collected the ultrasound data from the subject. The training data for the statistical model may include ultrasound data labeled with the pose of the ultrasound device relative to the subject when the ultrasound device collected the ultrasound data from the subject. In some embodiments, to determine the location and orientation of the ultrasound device relative to the subject for the training data, any of the methods described with reference to act 706 for determining the location and orientation of the ultrasound device relative to the subject may be used. The local processing device may then use this statistical model and the currently collected ultrasound data to determine the current location and orientation.

In some embodiments, the local processing device may associate the pose data with a time at which the pose data was collected. In some embodiments, the local processing device may associate the pose data with ultrasound data that was collected at the same time as the pose data. As described above, the pose data may pose data for the ultrasound device relative to the local processing or pose data for the ultrasound device relative to the subject. It should also be appreciated that in some embodiments, the local processing device may not generate pose data. The process 700 proceeds from act 706 to act 708.

In act 708, the local processing device transmits the sets of ultrasound data, or portions or indications thereof, to the remote processing device. If the local processing device transmits indications of the sets of ultrasound data to the remote processing device, the local processing device may transmit the sets of ultrasound data, or portions thereof, to a server, and the remote processing device may access the sets of ultrasound data from the server using the indications. In some embodiments, the local processing device may also transmit pose data, or portions or indications thereof, to the remote processing device. As described above, the pose data may the pose of the ultrasound device relative to the local processing or the pose of the ultrasound device relative to the subject. The process 700 proceeds from act 708 to act 710.

In act 710, the local processing device receives, from the remote processing device, an indication of a selected set of ultrasound data. For example, the remote expert may view, on the remote processing device, the sets of ultrasound data, and select a particular set of ultrasound data (e.g., a set of ultrasound data showing a target anatomical view). Upon receiving the selection of the set of ultrasound data, the remote processing device may transmit to the local processing device an indication of the selected set of data. In some embodiments, the indication of the selected set of data may be an identifier of the selected set of ultrasound data. In some embodiments, the indication of the selected set of data may be a timestamp indicating the time when the selected set of ultrasound data was collected. In some embodiments, the indication of the selected set of data may include pose data from the time when the selected set of data was collected. It should be appreciated that a remote expert need not necessarily specifically select a set of ultrasound data such that the set of ultrasound data is considered the selected set of ultrasound data. For example, the remote expert may select some other data, such as pose data, that is associated with a set of ultrasound data, and that set of ultrasound data may be considered the selected set of ultrasound data. The process 700 proceeds from act 710 to act 712.

In act 712, the local processing device provides an instruction for moving the ultrasound device to the position at which the selected set of ultrasound data was collected (this position may be referred to as the target pose). In some embodiments, the instruction may be based on the pose data collected when the selected set of ultrasound data was collected. In some embodiments, the local processing device may determine a time at which the selected set of ultrasound data was collected (e.g., using timestamps) and then retrieve the pose data collected at or approximately at that time. In some embodiments, pose data may be associated with each set of ultrasound data, and the local processing device may retrieve the pose data associated with the selected set of ultrasound data. In some embodiments, the remote processing device may transmit the pose data (either the pose of the ultrasound device relative to the subject or relative to the local processing device, as described above) associated with the selected set of ultrasound data to the local processing device.

In some embodiments, the local processing device may determine the current pose of the ultrasound device (e.g., using the methods described with reference to act 706), compare the current pose to the target pose, and provide an instruction for bringing the current pose closer to the target pose. In some embodiments, the poses used may be poses of the ultrasound device relative to the subject. In some embodiments, the poses used may be poses of the ultrasound device relative to the local processing device. In such embodiments, as described above, the local processing device may be stationary (e.g., the local processing device may be held by a holder such as a clamp) or the local processing device may instruct the user to hold the local processing device stationary. This may be helpful to avoid a situation in which the local processing device moves between act 706 and 712, such that a given pose of the ultrasound device relative to the local processing device may not necessarily be in the same position on the patient at act 706 and act 712. Then, the local processing device may provide an instruction for moving the ultrasound device in order to bring the local processing device closer to the target pose. The instruction may be expressed relative to the subject. For example, if the local processing device determines that the current pose of the ultrasound device is inferior relative to the subject from the target pose, the local processing device may provide an instruction to move the ultrasound device in the superior direction relative to the subject.

In some embodiments, a statistical model may be trained to determine, based on ultrasound data, a pose of the ultrasound device relative to the subject when the ultrasound device collected the ultrasound data from the subject. The training data for the statistical model may include ultrasound data labeled with the pose of the ultrasound device relative to the subject when the ultrasound device collected the ultrasound data from the subject. To determine the pose of the ultrasound device relative to the subject for the training data, any of the methods described with reference to act 706 for determining the pose of the ultrasound device relative to the local processing device, and any of the methods for determining the pose of the local processing device relative to the external world described with reference to act 712, may be used. The local processing device may then use this statistical model to determine a target pose based on the selected set of ultrasound data.

In some embodiments, a statistical model may be trained to accept ultrasound data as an input and output a set of coordinates in a coordinate system, where the coordinate system models an anatomical area and the outputted set of coordinates corresponds to the location within the anatomical area where the ultrasound data was collected. As a simplified example for illustration purposes only, the torso of a subject may be divided into a two-dimensional grid of 25 locations, with the location at the upper left of the grid having coordinates (0,0), the location at the upper right of the grid having coordinates (0,5), the location at the lower left of the grid having coordinates (5,0), and the location at the lower right of the grid having coordinates (5,5). To train the statistical model, multiple sets of ultrasound data each collected from a respective anatomical location may be labeled with that anatomical location's corresponding set of coordinates. The statistical model may thereby learn to determine, based on inputted ultrasound data, a set of coordinates corresponding to the ultrasound data. The local processing device may input the selected set of ultrasound data to the statistical model in order to determine the corresponding set of coordinates (referred to herein as the target set of coordinates). The local processing device may also input the ultrasound data collected by the ultrasound device at its current position to the statistical model in order to determine the corresponding set of coordinates (referred to herein as the current set of coordinates). The local processing device may determine the direction for moving the ultrasound device based on the target coordinates and the current coordinates. As an illustrative example, if the current set of coordinates is (0,5) and the target set of coordinates is (5,0), the local processing device may determine that the ultrasound device must be moved in the superior direction and then the rightwards direction (or the rightwards direction and then the superior direction) relative to the subject. As the user moves the ultrasound device, the local processing device may update the current set of coordinates based on newly collected ultrasound data and update the instruction (e.g., the direction for moving the ultrasound device) provided to the user. In some embodiments, at act 710, the local processing device may receive from the remote processing device the target set of coordinates corresponding to the selected set of ultrasound data. In other words, the remote processing device, rather than the local processing device, may determine the target set of coordinates.

In some embodiments, the local processing device may display the instruction as an arrow superimposed on a frame of video such that the arrow points in the direction relative to the subject as depicted in the video. For example, if the superior direction relative to the subject as depicted on the local processing display's display screen, the arrow may point to the right in order to provide an instruction to move the ultrasound device in the superior direction. Thus, the instruction may be part of an augmented reality interface on the local processing device, as video of the real world may be augmented by a non-real arrow superimposed on the video. In some embodiments, the local processing device may determine an arrow (or other directional indicator) to display as an instruction, translate and/or rotate and/or tilt that arrow in three-dimensional space based on the pose of the ultrasound device relative to the local processing device, and then project that three-dimensional arrow into two-dimensional space for display on the display screen of the local processing device. The local processing device may thus determine, based on the pose of the ultrasound device relative to the local processing device, the positioning of the arrow on the display screen and how the arrow appears to be rotated in three dimensions. The local processing device may use the same method to provide the instruction in act 704.

In some embodiments, the subject being imaged may be oriented in a default orientation relative to gravity. For example, the subject being imaged may be lying on his/her left side, such that moving the ultrasound device towards the subject's left side is in the direction of gravity, moving the ultrasound device towards the subject's head is 90 degrees relative to gravity, moving the ultrasound device toward the subject's right side is 180 degrees relative to gravity, and moving the ultrasound device towards the subject's legs is 270 degrees relative to gravity. Thus, an instruction for moving the ultrasound device relative to the subject may be converted to an instruction for moving the ultrasound device relative to gravity. If the ultrasound device is initially oriented in a known orientation relative to gravity, and the instruction for moving the ultrasound device is relative to gravity, then the local processing device may use the pose of the ultrasound device relative to the local processing device to determine how to display the instruction on the local processing device. The local processing device may instruct the user how to initially orient the ultrasound device in a known orientation relative to gravity based on motion and/or orientation data from the ultrasound device. After this, as the user changes the orientation of the ultrasound device (e.g., while following instructions), the local processing device may track the deviation of the ultrasound device from the initial orientation relative to gravity using motion and/or orientation data from the ultrasound device. The local processing device may use this tracked deviation to continue to determine how the ultrasound device is oriented relative to gravity and continue to determine how to display, on the local processing device, arrows relative to gravity. The process 700 proceeds from act 712 to act 716.

In act 716, the local processing device receives further ultrasound data at the position of the ultrasound device at which the selected set of ultrasound was collected (i.e., the target pose). When the ultrasound device is at the target position, the ultrasound device may be capable of collecting a target anatomical view. In some embodiments, the local processing device may receive, from the remote processing device, an instruction to collect the further ultrasound data at this position. In some embodiments, the local processing device may automatically collect the further ultrasound data at this position. In some embodiments, the local processing device may automatically instruct the user to collect the further ultrasound data at this position. The local processing device may receive more ultrasound data (e.g., more frames of ultrasound images and/or ultrasound data spanning a longer time period) at act 716 than the local processing device received when the ultrasound data was at the target position in act 706. For example, in act 706, the local processing device may have received from the ultrasound device ultrasound data spanning a portion of a heart cycle while the ultrasound device was at the target position. At act 716, the local processing device may receive from the ultrasound device ultrasound data spanning one or more complete heart cycles while the ultrasound device is at the target position. Thus, the local processing device may instruct the user at act 716 to maintain the ultrasound device in its current position for a specific period of time. The period of time may be a default period of time, a time selected by the remote expert, or the remote expert may transmit an instruction from the remote processing device to the local processing device instructing the user to cease collection of ultrasound data.

In some embodiments, prior to act 702, the local processing device may provide an instruction to the user to move the ultrasound device to a default rotation and a default tilt on the subject. In some embodiments, the local processing device may receive the instruction from the remote processing device. In some embodiments, the local processing device may automatically generate or retrieved the instruction. In some embodiments, once the ultrasound device is at the default orientation, the process 700 may proceed through an iteration of acts 702-712, where the multiple positions may be multiple locations of the ultrasound device on the subject while the ultrasound device is maintained at the default rotation and the default tilt. The position at which the selected set of ultrasound data was collected may be the target location, default rotation, and default tilt. Based on the instruction provided in act 712, the user may move the ultrasound device to this position. The process 700 may then proceed through another iteration of acts 702-712. In this iteration, the multiple positions may be multiple rotations of the ultrasound device on the subject while the ultrasound device is maintained at the target location and the default tilt. The position at which the selected set of data was collected may be the target location, target rotation, and default tilt. Based on the instruction provided in act 712, the user may move the ultrasound device to this position. The process 700 may then proceed through another iteration of acts 702-712. In this iteration, the multiple positions may be multiple tilts of the ultrasound device on the subject while the ultrasound device is maintained at the target location and the target rotation. The position at which the selected set of data was collected may be the target location, target rotation, and target tilt, in other words, the target position. The process 700 may then proceed to act 716. It should be appreciated that while the above description included first providing instructions to move the ultrasound device to the target location, then to the target rotation, and then to the target tilt, instructions may be provided in other orders (e.g., rotation then tilt then location, tilt then orientation then location, etc.).

Thus, at act 712, the process 700 may either proceed back to act 702 or proceed to act 716. In some embodiments, a remote expert operating the remote processing device may determine whether to proceed back to act 702 or to proceed to act 716. In such embodiments, the local processing device may then receive from the remote processing device either an instruction to collect sets of ultrasound data from multiple positions (act 702) or an instruction to collect further ultrasound data at the position of the ultrasound device at which the selected set of ultrasound was collected (as a precursor to act 706). The local processing device may also transmit ultrasound data from the current position to the remote processing device. In some embodiments, the instruction to collect further ultrasound data at the position of the ultrasound device at which the selected set of ultrasound was collected may be an explicit instruction to the user (e.g., provided on a display screen or from speakers) to collect the further ultrasound data. In some embodiments, the instruction to collect further ultrasound data at the position of the ultrasound device at which the selected set of ultrasound was collected may be a command to the ultrasound device to automatically collect the further ultrasound data.

In some embodiments, the local processing device may determine whether to proceed back to act 702 or to proceed to act 716. In some embodiments, the local processing device may use a statistical model trained to determine whether ultrasound data contains a target anatomical view. The statistical model may be trained on ultrasound data labeled with whether it contains a target anatomical view or not. When the ultrasound device is at the position at which the selected set of ultrasound was collected, the statistical model may determine if the ultrasound data collected at this position does not contain the target anatomical view. If it does not contain the target anatomical view, in some embodiments the local processing device may wait to receive from the remote processing device an instruction to collect sets of ultrasound data from multiple positions (i.e., to proceed back to act 702). In some embodiments, the local processing device may provide a prompt to the remote processing device that prompts the remote expert to provide an instruction to collect sets of ultrasound data from multiple positions (i.e., to proceed back to act 702). The local processing device may also transmit ultrasound data from the current position to the remote processing device. If ultrasound data collected at the current position does not contain the target anatomical view, in some embodiments the local processing device may provide an explicit instruction to collect further ultrasound data at the position of the ultrasound device at which the selected set of ultrasound was collected (as a precursor to act 716). In some embodiments, the local processing device may automatically collect further ultrasound data at the position of the ultrasound device at which the selected set of ultrasound was collected (act 716).

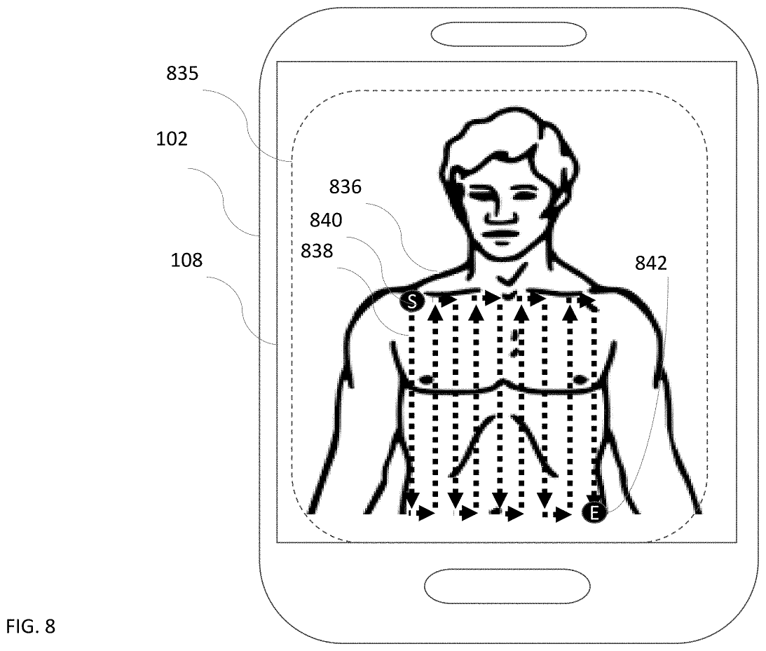

FIG. 8 illustrates an example instruction that may be provided by the processing device 102, in accordance with certain embodiments described herein. The instruction may be the instruction provided in act 706, or a portion thereof. The instruction includes an image 835 displayed by the display screen 108 of the local processing device 102 and depicting a subject 836, a path 838, a start point 840, and an end point 842. The path 838 is a serpentine path covering substantially all of the torso of the subject 836 and extending from the start point 840 at the top right corner of the torso (from the view of the subject 836) to the end point 842 at the bottom left corner of the torso.

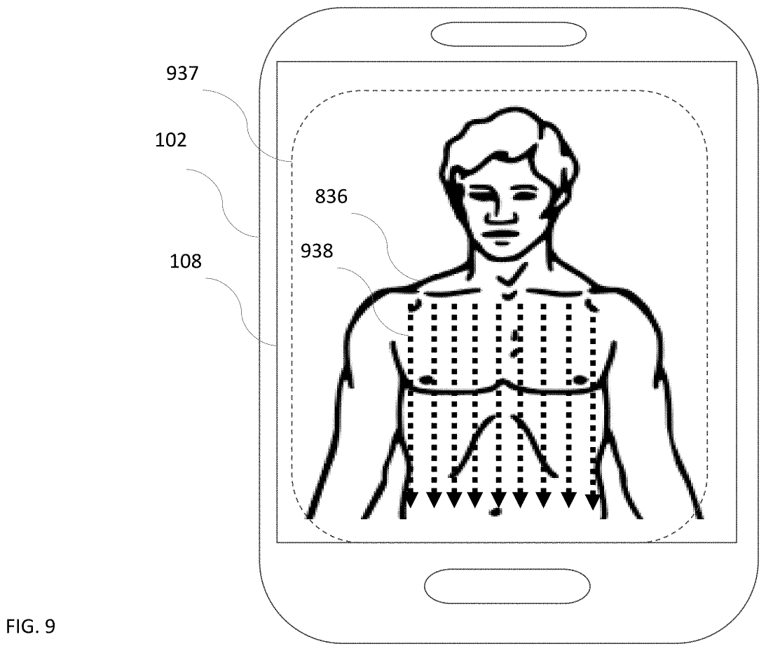

FIG. 9 illustrates another example instruction that may be provided by the processing device 102, in accordance with certain embodiments described herein. The instruction may be the instruction provided in act 706, or a portion thereof. The instruction includes an image 937 displayed by the display screen 108 of the local processing device 102 and depicting the subject 836 and a path 938. The path 938 covers substantially all of the torso of the subject 836 and extends in parallel legs all proceeding in substantially the same direction across the torso.

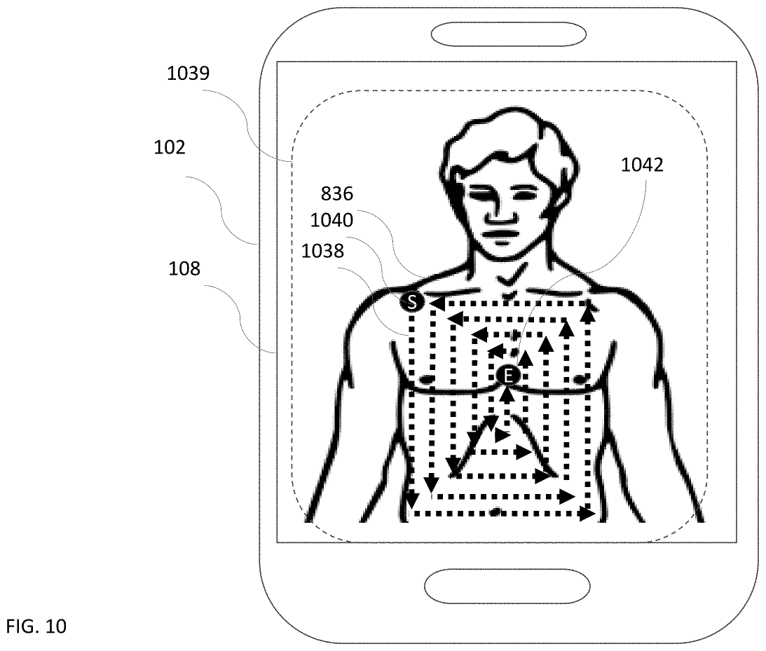

FIG. 10 illustrates another example instruction that may be provided by the processing device 102, in accordance with certain embodiments described herein. The instruction may be the instruction provided in act 706, or a portion thereof. The instruction includes an image 1039 displayed by the display screen 108 of the local processing device 102 and depicting the subject 836, a path 1038, a start point 1040, and an end point 1042. The path 1038 is a spiral path covering substantially all of the torso of the subject 836 and extending from the start point 1040 at the top right corner of the torso (from the view of the subject 836) to the end point 1042 at the center of the torso.

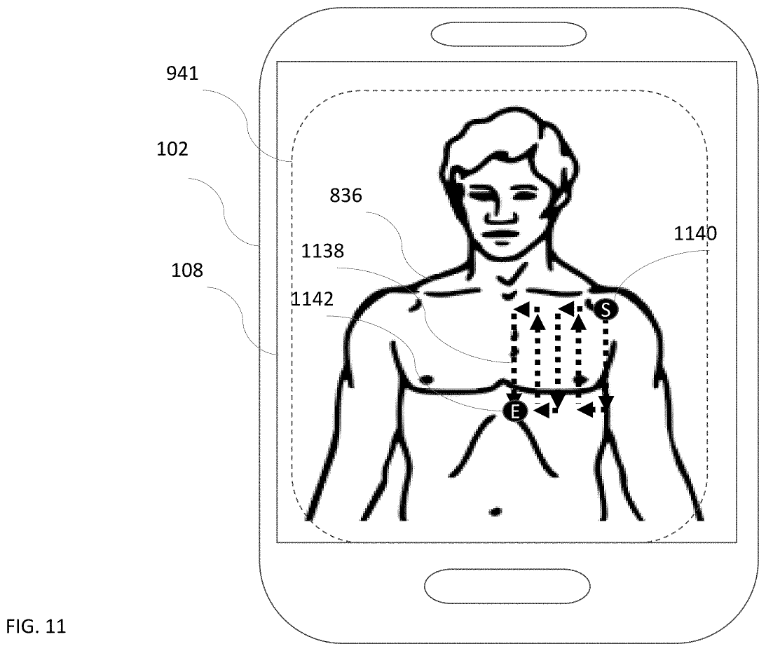

FIG. 11 illustrates another example instruction that may be provided by the processing device 102, in accordance with certain embodiments described herein. The instruction may be the instruction provided in act 706, or a portion thereof. The instruction includes an image 1141 displayed by the display screen 108 of the local processing device 102 and depicting the subject 836, a path 1138, a start point 1140, and an end point 1142. The path 1138 is a serpentine path covering substantially all of the upper left portion of the torso of the subject 836 (from the view of the subject 836) and extending from the start point 1140 at the top left corner of the torso to the end point 1142 at center of the torso. The path 1138 is similar to the path 838, except that the path 1138 covers a different anatomical area.

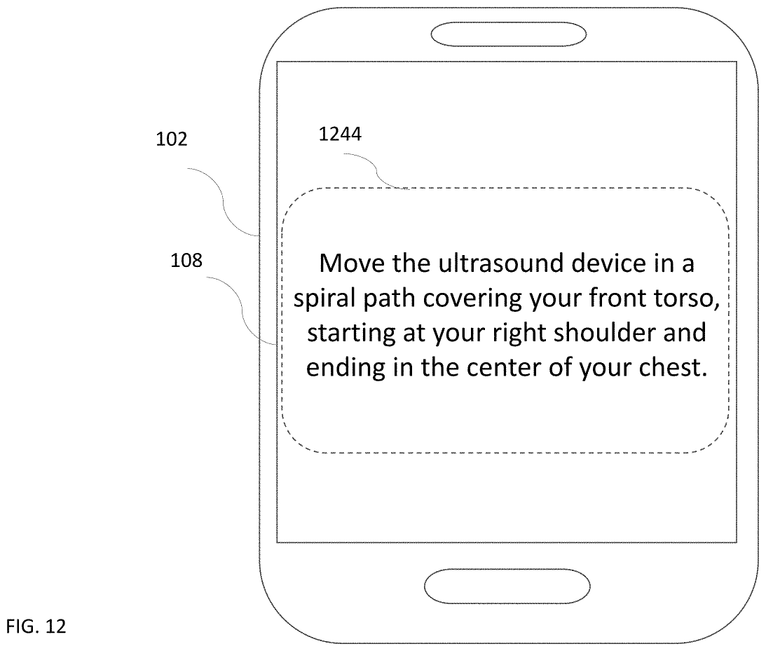

FIG. 12 illustrates another example instruction that may be provided by the processing device 102, in accordance with certain embodiments described herein. The instruction may be the instruction provided in act 706, or a portion thereof. The instruction includes text 1244 displayed by the display screen 108 of the processing device 102. The text 1244 instructs the user to move the ultrasound device in a spiral path covering the subject's front torso, starting at the right shoulder and ending in the center of the chest.

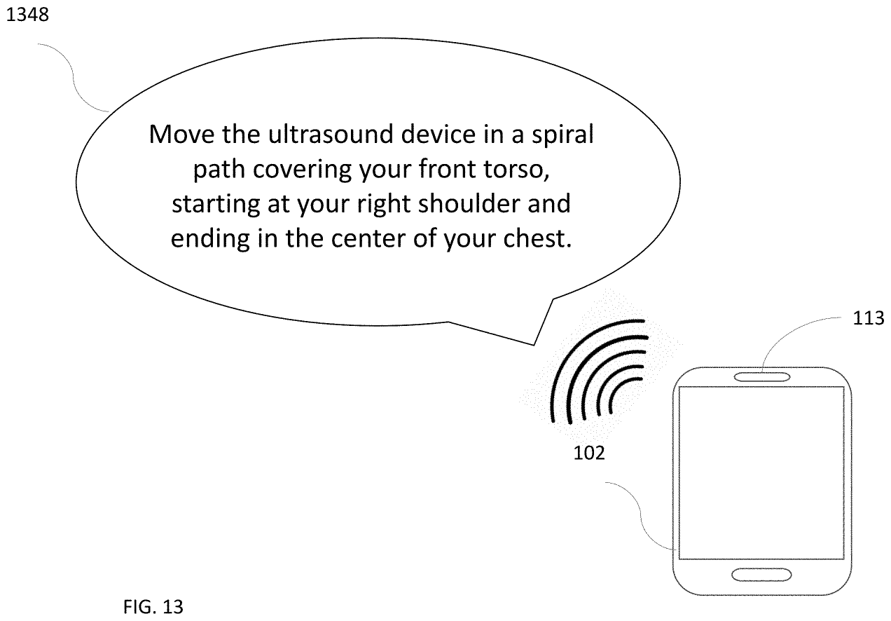

FIG. 13 illustrates another example instruction that may be provided by the processing device 102, in accordance with certain embodiments described herein. The instruction may be the instruction provided in act 706, or a portion thereof. The instruction includes audio 1348 output by a speaker 113 of the processing device 102. The audio 1348 instructs the user to move the ultrasound device in a spiral path covering the subject's front torso, starting at the right shoulder and ending in the center of the chest.

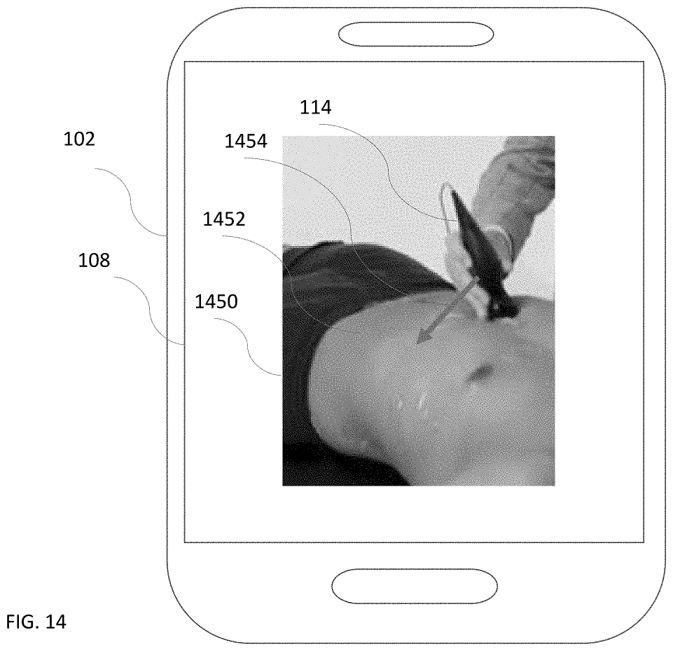

FIG. 14 illustrates another example instruction that may be provided by the processing device 102, in accordance with certain embodiments described herein. The instruction may be the instruction provided in act 712, or a portion thereof. The instruction includes a frame of video 1450 depicting the subject 1452 and the ultrasound device 114. Superimposed on the frame of video 1450 is an arrow 1454 indicating a direction for moving the ultrasound device 114 to the target location.

The example instructions described and shown herein are non-limiting, and it should be understood that instructions having other forms and content (e.g., different texts) may also be used. For example, instructions for other anatomical areas besides the torso, as appropriate, may be used. Instructions to move the ultrasound device in a path having different forms (e.g., spiral, serpentine, or some other form) may be used. The instructions may also include other content besides what is described and shown. For example, an instruction may include both an image and text, or a video and text, etc. As another example, while an instruction is provided, the most recently collected ultrasound image may also be shown on the display screen of the processing device. As another examples, an instruction to maintain the ultrasound device at its current rotation and/or current tilt may also be provided.

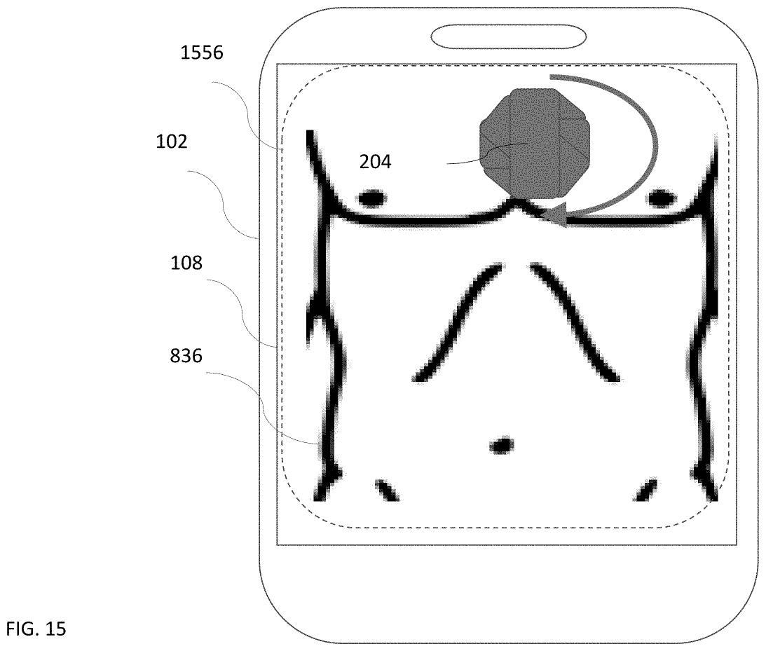

FIG. 15 illustrates an example instruction that may be provided by the processing device 102, in accordance with certain embodiments described herein. The instruction may be the instruction provided in act 706, or a portion thereof. The instruction includes an image 1556 displayed by the display screen 108 of the local processing device 102. The image 1556 shows multiple stages of an ultrasound device (as represented from a bird's eye view by an outline of the sensor 204 of the ultrasound device) being rotated about a location.

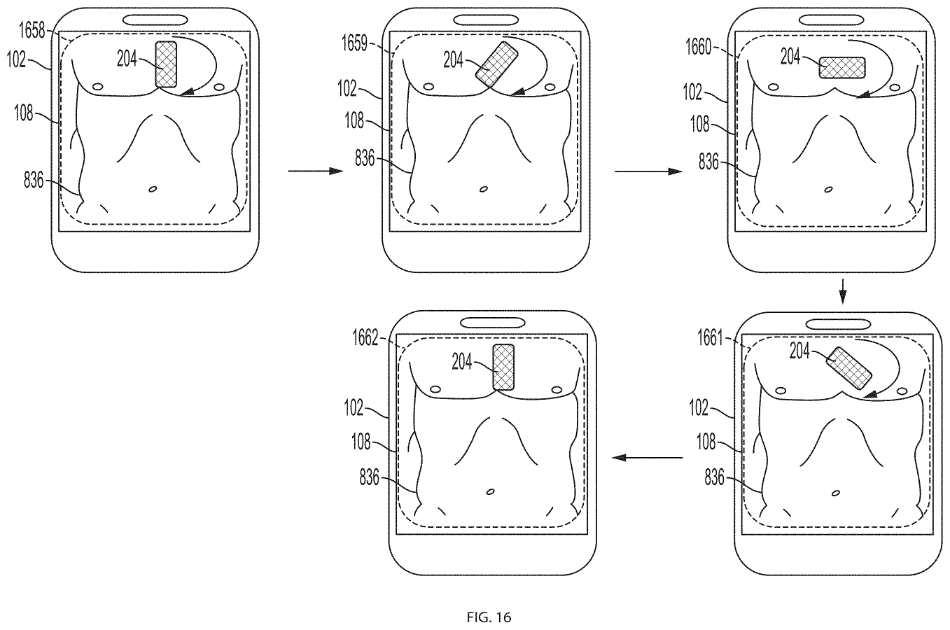

FIG. 16 illustrates another example instruction that may be provided by the processing device 102, in accordance with certain embodiments described herein. The instruction may be the instruction provided in act 706, or a portion thereof. The instruction includes video displayed by the display screen 108 of the processing device 102. The video includes multiple frames 1658-1662. The frames 1658-1662 show multiple stages of an ultrasound device (as represented from a bird's eye view by an outline of the sensor 204 of the ultrasound device) being rotated about a location.

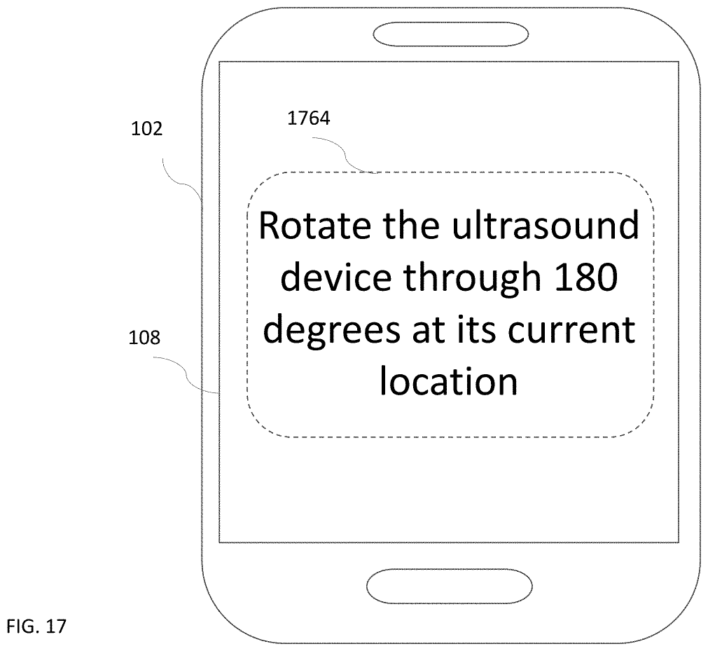

FIG. 17 illustrates another example instruction that may be provided by the processing device 102, in accordance with certain embodiments described herein. The instruction may be the instruction provided in act 706, or a portion thereof. The instruction includes text 1764 displayed by the display screen 108 of the processing device 102. The text 1764 instructs the user to rotate the ultrasound device through 180 degrees at its current location.

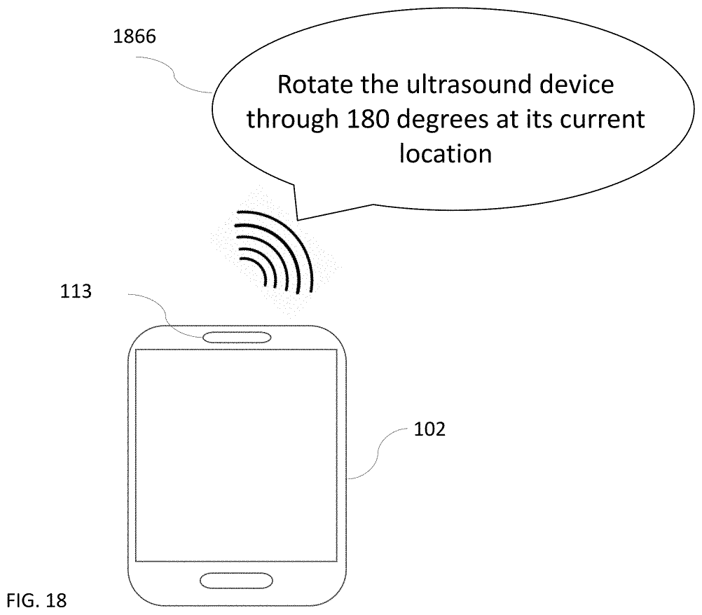

FIG. 18 illustrates another example instruction that may be provided by the processing device 102, in accordance with certain embodiments described herein. The instruction may be the instruction provided in act 706, or a portion thereof. The instruction includes audio 1866 output by the speaker 113 of the processing device 102. The audio 1866 instructs the user to rotate the ultrasound device through 180 degrees at its current location.

The example instructions described and shown herein are non-limiting, and it should be understood that instructions having other forms and content (e.g., different texts) may also be used. Instructions to rotate the ultrasound device through a different number of degrees than 180 may be used. The instructions may also include other content besides what is described and shown. For example, an instruction may include both an image and text, or a video and text, etc. As another example, while an instruction is provided, the most recently collected ultrasound image may be shown on the display screen of the processing device. As another example, an instruction to maintain the ultrasound device at its current location and/or current tilt may also be provided.

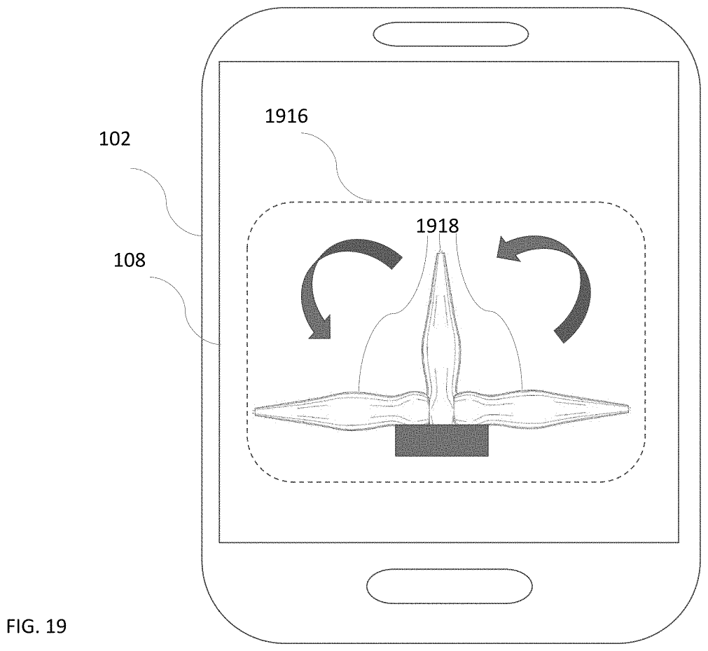

FIG. 19 illustrates an example instruction that may be provided by the processing device 102, in accordance with certain embodiments described herein. The instruction may be the instruction provided in act 706, or a portion thereof. The instruction includes an image 1916 displayed by the display screen 108 of the local processing device 102. The image 1916 shows multiple stages of an ultrasound device 1918 being tilted about a location.

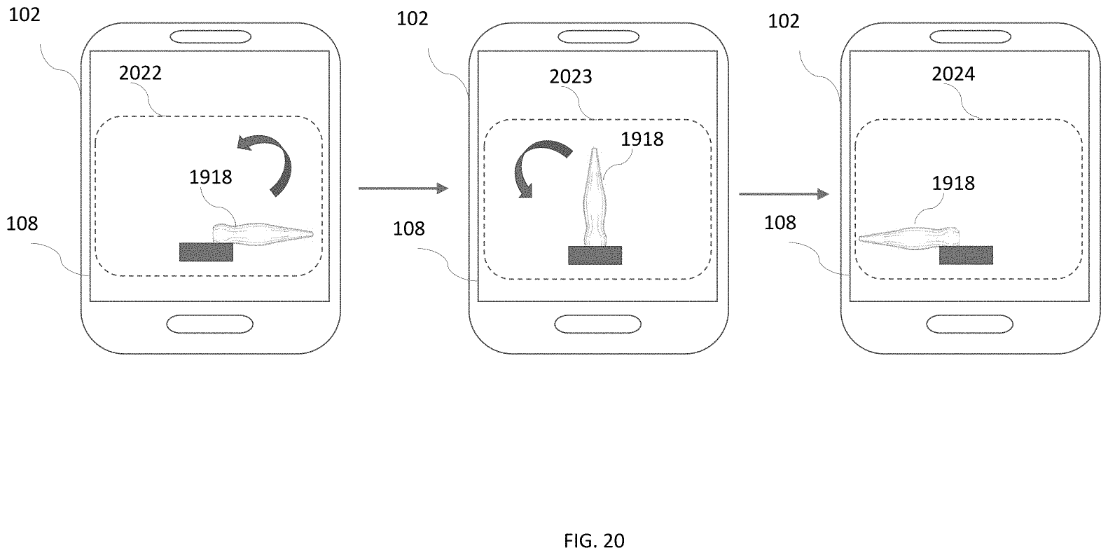

FIG. 20 illustrates another example instruction that may be provided by the processing device 102, in accordance with certain embodiments described herein. The instruction may be the instruction provided in act 706, or a portion thereof. The instruction includes video displayed by the display screen 108 of the processing device 102. The video includes multiple frames 2022-2024. The video shows multiple stages of an ultrasound device 1918 being tilted about a location.

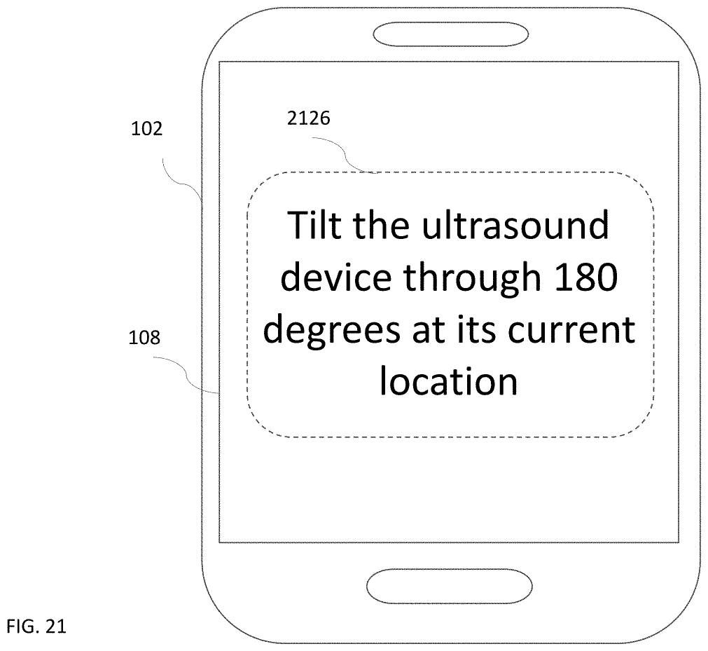

FIG. 21 illustrates another example instruction that may be provided by the processing device 102, in accordance with certain embodiments described herein. The instruction may be the instruction provided in act 706, or a portion thereof. The instruction includes text 2126 displayed by the display screen 108 of the processing device 102. The text 2126 instructs the user to tilt the ultrasound device through 180 degrees at its current location.

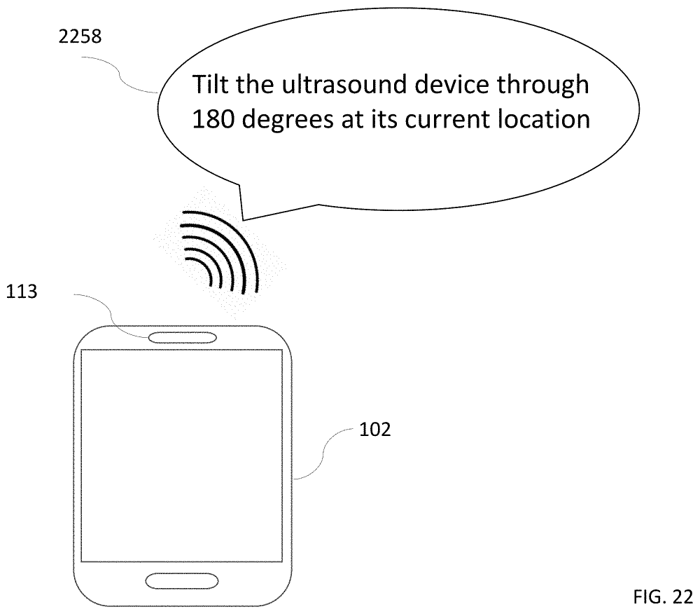

FIG. 22 illustrates another example instruction that may be provided by the processing device 102, in accordance with certain embodiments described herein. The instruction may be the instruction provided in act 706, or a portion thereof. The instruction includes audio 2228 output by a speaker 113 of the processing device 102. The audio 2228 instructs the user to tilt the ultrasound device through 180 degrees at its current location.

The example instructions described and shown herein are non-limiting, and it should be understood that instructions having other forms and content (e.g., different texts) may also be used. Instructions to tilt the ultrasound device through a different number of degrees than 180 may be used. The instructions may also include other content besides what is described and shown. For example, an instruction may include both an image and text, or a video and text, etc. As another example, while an instruction is provided, the most recently collected ultrasound image may be shown on the display screen of the processing device. As another examples, an instruction to maintain the ultrasound device at its current rotation and/or current rotation may also be provided.

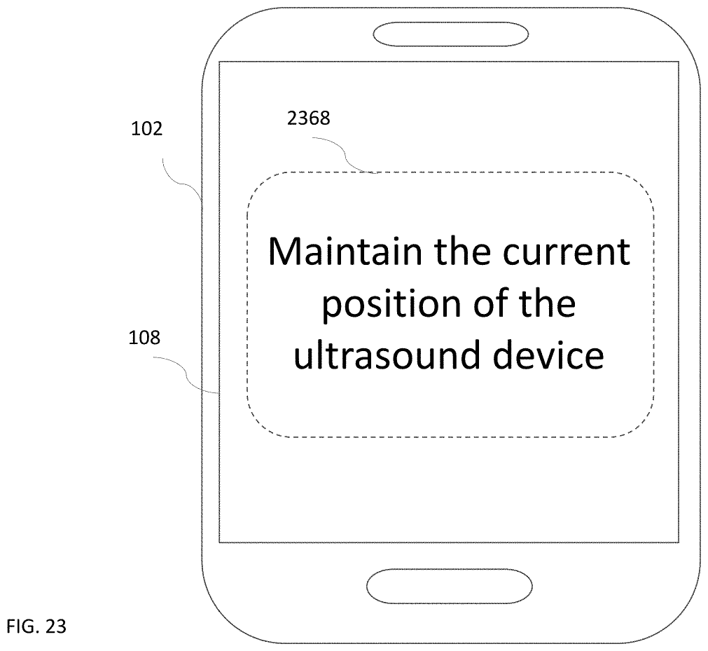

FIG. 23 illustrates another example instruction that may be provided by the local processing device 102, in accordance with certain embodiments described herein. The instruction may be the instruction provided in act 712, or a portion thereof. The instruction instructs the user to maintain the current position of the ultrasound device. In some embodiments, the user may be moving the ultrasound device to a target location, target rotation, and/or target tilt, based on instruction provided in act 712. Once the local processing device determines that the ultrasound device is at the target location, target rotation, and/or target tilt, the local processing device may provide the instruction illustrated in FIG. 23 such that the user stops moving the ultrasound device. If the ultrasound device is at the target position, the ultrasound device may collect further ultrasound data, as described above with reference to act 716. If the ultrasound device is not at the target position, acts 702-712 may be performed again. For example, if the ultrasound device has been translated to the target location but is not at the target rotation or the target tilt, the local processing device may receive and provide the instruction of FIG. 23 to instruct the user to stop translating the ultrasound device, and then proceed back to act 702 to receive a new instruction. In FIG. 23, the instruction includes text 2368 displayed by the display screen 108 of the processing device 102. The text 2368 instructs the user to maintain the current position of the ultrasound device.

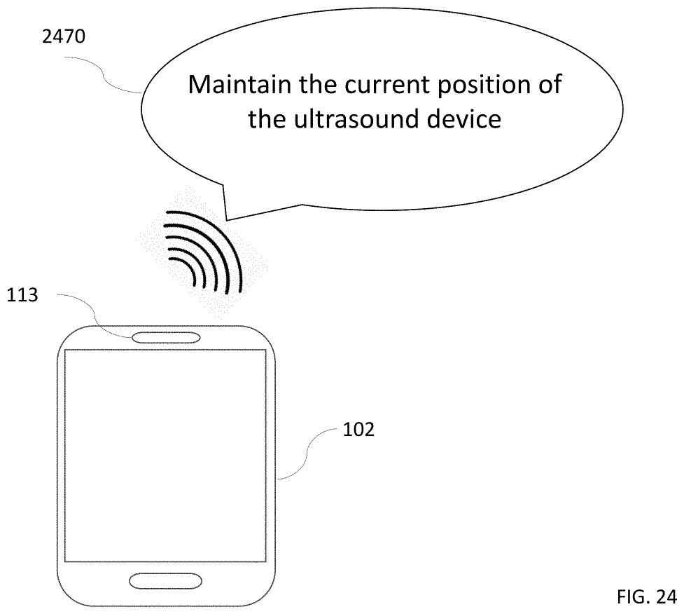

FIG. 24 illustrates another example instruction that may be provided by the processing device 102, in accordance with certain embodiments described herein. The instruction may be the instruction provided in act 712, or a portion thereof. The instruction in FIG. 24 is similar to the instruction in FIG. 23, except that rather than providing the instruction through text, the instruction of FIG. 24 includes audio 2470 output by a speaker 113 of the processing device 102. The audio 2470 instructs the user to maintain the current position of the ultrasound device.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A method, comprising:
providing, by a first processing device in operative communication with an ultrasound device, an instruction to collect sets of ultrasound data from multiple positions of the ultrasound device;
receiving, from the ultrasound device, the sets of ultrasound data;
transmitting the sets of ultrasound data, or portions or indications thereof, to a second processing device;
receiving, from the second processing device, an indication of a selected set of ultrasound data;
providing an instruction to move the ultrasound device to a position at which the selected set of ultrasound data was collected; and
receiving further ultrasound data from the ultrasound device at the position at which the selected set of ultrasound data was collected.

2. The method of claim 1, wherein:
providing the instruction to collect the sets of ultrasound data from the multiple positions of the ultrasound device comprises providing an instruction to collect sets of ultrasound data from multiple locations of the ultrasound device;
each of the sets of ultrasound data includes ultrasound data collected at a particular location of the ultrasound device; and
providing the instruction to move the ultrasound device to the position at which the selected set of ultrasound data was collected comprises providing an instruction to translate the ultrasound device to a location of the ultrasound device at which the selected set of ultrasound data was collected.

3. The method of claim 2, wherein providing the instruction to collect the sets of ultrasound data from the multiple locations of the ultrasound device comprises providing an instruction to move the ultrasound device across substantially all of an anatomical area.

4. The method of claim 3, wherein the anatomical area is greater than 25 cm² in area.

5. The method of claim 2, wherein providing the instruction to collect the sets of ultrasound data from the multiple locations of the ultrasound device comprises providing an instruction to move the ultrasound device in a serpentine path.

6. The method of claim 2, wherein providing the instruction to collect the sets of ultrasound data from the multiple locations of the ultrasound device comprises providing an instruction to move the ultrasound device in a spiral path.

7. The method of claim 2, wherein providing the instruction to collect the sets of ultrasound data from the multiple locations of the ultrasound device comprises providing an instruction to maintain the ultrasound device at its current rotation and/or its current tilt while translating the ultrasound device.

8. The method of claim 2, wherein providing the instruction to translate the ultrasound device to the location of the ultrasound device at which the selected set of ultrasound data was collected comprises providing an instruction to maintain the ultrasound device at its current rotation and/or its current tilt while translating the ultrasound device.

9. The method of claim 1, wherein:
providing the instruction to collect the sets of ultrasound data from the multiple positions of the ultrasound device comprises providing an instruction to collect sets of ultrasound data from multiple rotations of the ultrasound device;
each of the sets of ultrasound data includes ultrasound data collected at a particular rotation of the ultrasound device; and
providing the instruction to move the ultrasound device to the position at which the selected set of ultrasound data was collected comprises providing an instruction to rotate the ultrasound device to a rotation of the ultrasound device at which the selected set of ultrasound data was collected.

10. The method of claim 9, wherein providing the instruction to collect the sets of ultrasound data from the multiple rotations of the ultrasound device comprises providing an instruction to rotate the ultrasound device between approximately 85 degrees and 95 degrees about a location.

11. The method of claim 9, wherein providing the instruction to collect the sets of ultrasound data from the multiple rotations of the ultrasound device comprises providing an instruction to rotate the ultrasound device between approximately 175 degrees and 185 degrees about a location.

12. The method of claim 9, wherein providing the instruction to collect the sets of ultrasound data from the multiple rotations of the ultrasound device comprises providing an instruction to rotate the ultrasound device between approximately 355 degrees and 365 degrees about a location.

13. The method of claim 9, wherein providing the instruction to collect the sets of ultrasound data from the multiple rotations of the ultrasound device comprises providing an instruction to maintain the ultrasound device at its current location and/or its current tilt while rotating the ultrasound device.

14. The method of claim 9, wherein providing the instruction to rotate the ultrasound device to the rotation of the ultrasound device at which the selected set of ultrasound data was collected comprises providing an instruction to maintain the ultrasound device at its current location and/or its current tilt while rotating the ultrasound device.

15. The method of claim 1, wherein:
providing the instruction to collect the sets of ultrasound data from the multiple positions of the ultrasound device comprises providing an instruction to collect sets of ultrasound data from multiple tilts of the ultrasound device;
each of the sets of ultrasound data includes ultrasound data collected at a particular tilt of the ultrasound device; and
providing the instruction to move the ultrasound device to the position at which the selected set of ultrasound data was collected comprises providing an instruction to move the ultrasound device to a tilt of the ultrasound device at which the selected set of ultrasound data was collected.

16. The method of claim 15, wherein providing the instruction to collect the sets of ultrasound data from the multiple tilts of the ultrasound device comprises providing an instruction to tilt the ultrasound device between approximately 85 degrees and 95 degrees about a location.

17. The method of claim 15, wherein providing the instruction to collect the sets of ultrasound data from the multiple tilts of the ultrasound device comprises providing an instruction to tilt the ultrasound device approximately 180 degrees about a location.

18. The method of claim 15, wherein providing the instruction to collect the sets of ultrasound data from the multiple tilts of the ultrasound device comprises providing an instruction to maintain the ultrasound device at its current location and/or its current rotation while tilting the ultrasound device.

19. The method of claim 15, wherein providing the instruction to tilt the ultrasound device to the tilt of the ultrasound device at which the selected set of ultrasound data was collected comprises providing an instruction to maintain the ultrasound device at its current location and/or its current rotating while tilting the ultrasound device.

20. The method of claim 1, further comprising:
receiving the instruction to collect the sets of ultrasound data from the multiple positions of the ultrasound device from the second processing device.
